(19) United States
(12) Patent Application Publication
(10) Pub. No.: US 2004/0209811 A1
(43) Pub. Date: Oct. 21, 2004

(54) Oral prohylactic agent against viral infection

(75) Inventor: Chuang Chun Chiueh, Taipei (TW)

Correspondence Address: BACON & THOMAS, PLLC, 625 SLATERS LANE, FOURTH FLOOR, ALEXANDRIA, VA, 22314

(73) Assignee: Chuang-Chun Chiueh, Taipei City (TW)

(21) Appl. No.: 10/785,985

(22) Filed: Feb. 26, 2004

(30) Foreign Application Priority Data
Feb. 26, 2003 (TW) 92104102

(51) Int. Cl. A61K 38/16

(52) U.S. Cl. 514/12; 514/6

(57) ABSTRACT

The present invention relates to an oral prophylactic agent against viral infection comprising water-soluble formula and/or enteric-coated formula, which includes ingredients selected from at least one of the group consisting of C-phycocyanin, allophycocyanin, spirulina growth factor, and combinations thereof.

### BACKGROUND OF THE INVENTION

1. Field of the Invention

The present invention discloses an oral prophylactic agent against viral infection comprising enteric-coated formula and/or water-soluble formula which comprises ingredients selected from any of the group consisting of C-phycocyanin (C-PC), allophycocyanin (APC), spirulina growth factor (SGF) and combinations thereof.

2. Description of Related Art

Enterovirus refers to a group of viruses that cause pathological changes of intestinal tract and belongs to a RNA viruse family Picornaviridae. The human intestinal tract cells are natural host of enterovirus. However, enterovirus is also found in other environments, such as wastewater, swimming pool, food and soil. Enteroviral infection is active in summer and autumn seasons and prone to attack children under age 15. It is transmitted through feces or respiratory secretions. Infected people usually show symptoms after 2-10 days of incubation period. In fact the majority of people infected with enterovirus (50-80%) experience either no symptoms or flu like symptoms. But some patients show special clinical manifestations, such as herpetic gingivostomatitis, hand-foot-mouth disease, aseptic meningitis, viral encephalitis, flaccid paralysis syndrome, acute myocarditis, and acute hemorrhagic conjunctivitis, wherein the first two are the most commonly seen.

Influenza is a contagious disease caused by virus. Such viruses are a group of RNA viruses belonging to family Orthomyxoviridae. By the serum immuno-reaction, influenza virus are designated as Types A, B and C. Influenza is prevalent in winter or early spring. The viruses invade the lung through oral or nasal routes and spread most easily in crowded places. The strains of influenza viruses differ each year. Chief symptoms of influenza include fever and soreness. People with influenza are usually bed ridden for 3-5 days and recover within 1-2 weeks.

According to U.S. Pat. No. 6,346,408 and other literature (master thesis of Tsai Kun-nan, Basic Medicine Research Institute of Chang Gung University), phycocyanin, a group of water soluble protein specifically found in algae is capable of inhibiting the reproduction of enterovirus and influenza virus. The mechanisms of phycocyanin action include: 1. preventing infection; and 2. delaying the reproduction of viruses in infected cells. Experiments demonstrate that phycocyanin at >0.3 μM concentration could fully prevent enteroviral infection, and such concentration was non-toxic to cells and effectively protected cells from viral invasion and pathological changes. Phycocyanin is also found to prevent the infection of influenza viruses at concentration >0.04 μM. Moreover, it is more effective in protecting cells from viruses by adding phycocyanin before the infection.

Spirulina growth factor (SGF) is the essence of blue green algae, which contains nucleic acids, nucleotides, small molecule proteins, sulfur-containing polysaccharides, water-soluble vitamins and minerals. SGF is rich in polysaccharides which can boost immune system and possess anti-viral and anti-tumor activities. Sulfur-containing polysaccharides are found by Harvard University to be effective against AIDS and many other viruses. (Journal of Phycology 1993; 29:125-130, Journal of Acquired Immune Deficiency Syndromes and Human Retrovirology, 1998; 18:7-12, International Immunopharmacology. 2002; 2:423-434).

### SUMMARY OF THE INVENTION

In light of the transmission routes of enterovirus and influenza virus, the present invention provides an oral prophylactic agent against viral infection. In one aspect, the foregoing oral prophylactic agent comprises water-soluble formula and/or enteric-coated formula. The foregoing water-soluble formula and enteric-coated formula can be present in the oral prophylactic agent separately or in combination.

The ingredients of the foregoing water-soluble formula can be selected from the group consisting of C-phycocyanin, allophycocyanin, spirulina growth factor, and combinations thereof.

The ingredients of the foregoing enteric-coated formula can be selected from the group consisting of C-phycocyanin, allophycocyanin, spirulina growth factor, and combinations thereof.

Specifically, the aforesaid oral prophylactic agent against viral infection comprises the combination of water-soluble formula and enteric-coated formula in which the ratio of water-soluble formula to enteric-coated formula is preferably 1:1˜1:10 by weight. , and more preferably 2:9.8 by weight. The ingredients of the foregoing water-soluble formula can be selected from the group consisting of C-phycocyanin, allophycocyanin, spirulina growth factor, and combinations thereof. The ingredients of the foregoing enteric-coated formula can be selected from the group consisting of C-phycocyanin, allophycocyanin, spirulina growth factor, and combinations thereof.

In an embodiment, the aforesaid oral prophylactic agent against viral infection comprises the combination of water-soluble formula and enteric-coated formula, both of which contain the combination of: C-phycocyanin, allophycocyanin, and spirulina growth factor The ratio of water-soluble formula to enteric-coated formula is preferably 1:1˜1:10 by weight, most preferably 2:9.8 by weight. Particularly, the percentage of three ingredients of the foregoing water-soluble formula is preferably: 3˜45% C-phycocyanin, 1˜15% allophycocyanin, and 96˜40% spirulina growth factor. More particularly, the aforesaid enteric-coated formula includes water-soluble formula, solid additive and vegetable oil, preferably in the percentage of: 10˜30% water-soluble formula, 15% solid additive and 75˜55% vegetable oil. The enteric-coated formula can be produced by granulation process. The water-soluble formula within aforesaid enteric-coated formula preferably comprises 3˜45% C-phycocyanin, 1˜15% allophycocyanin, and 96˜40% spirulina growth factor.

In the oral prophylactic agent of the present invention, the foregoing C-phycocyanin and allophycocyanin are collectively termed “phycocyanin” with concentration ranging from 25˜2000 μg/mL; the concentration of foregoing spirulina growth factor ranges from 1˜5000 μg/mL. The most preferable ratio of phycocyanin to spirulina growth factor in aforesaid oral prophylactic agent is 2:7.

The aforesaid oral prophylactic agent against viral infection can also comprises only water-soluble formula or enteric-coated formula, both of which contain the combination of C-phycocyanin, allophycocyanin, and spirulina growth factor, wherein the percentage of those three ingredients is: 3˜45% C-phycocyanin, 1˜15% allophycocyanin, and 96˜40% spirulina growth factor. The foregoing C-phycocyanin and allophycocyanin are collectively termed “phycocyanin” with concentration ranging between 25˜2000 μg/mL; the concentration of foregoing spirulina growth factor ranges between 1˜5000 μg/mL. The most preferable ratio of phycocyanin to spirulina growth factor in aforesaid oral prophylactic agent is 2:7.

The aforesaid water-soluble formula can be water-soluble powder, granule or any other water-soluble form.

The aforesaid enteric-coated formula can be enteric-coated granule, tablet, capsule or any other enteric-coated form.

In another aspect, the present invention is to provide an oral composition for prevention of viral infection comprising the aforementioned oral prophylactic agent. The foregoing oral composition can be added in food or drink, for example, but not limited to milk, yogurt, jelly, candy, chewing gum, lozenge or syrup. The foregoing oral composition works in a manner described below: By providing the oral mucosa environment with specific concentration of phycocyanin from the water-soluble formula of said oral agent, the infection of oral mucosal cells by enterovirus or influenza virus can be prevented, and moreover, the spirulina growth factor in the oral agent is rich in polysaccharides which can boost immunity against viruses. Plus, the enteric-coated formula of the oral agent can protect phycocyanin from being degraded by stomach acid and release its active ingredients only after reaching intestinal tract, wherein a specific concentration of phycocyanin is provided to inhibit the attack and reproduction of virus, thus achieving the invention of viral infection. The aforesaid water-soluble formula and enteric-coated formula may be present in the oral prophylactic agent separately or in combination based on actual needs to achieve the desired protective effect.

### DETAILED DESCRIPTION OF THE INVENTION

The following examples are presented in order to more fully illustrate the preferred embodiments of the invention. They should in no way be construed, however, as limiting the broad scope of the invention. While the invention is described and illustrated herein by references to various specific material, procedures and examples, it is understood that the invention is not restricted to the particular material combinations of material, and procedures selected for that purpose. Numerous variations of such details can be implied as will be appreciated by those skilled in the art.

### EXAMPLE 1

### Preparation of Oral Prophylactic Agent Against Viral Infection

(1) Ingredients and Proportions of Water-Soluble Formula (in Powder):

TABLE 1
Ingredients of water-soluble formula (in
powder) and their percentages

| Ingredient | Percentage |
|---|---|
| Allophycocyanin | 1˜15% |
| C-phycocyanin | 3˜45% |
| Spirulina growth factor | 96˜40% |

(2) Ingredients of Enteric-Coated Formula (in Granule) and Their Percentages:

TABLE 2
Ingredients of enteric-coated formula (in
granule) and their percentages

| Ingredient | Percentage |
|---|---|
| Water-soluble formula | 10˜30% |
| (in powder) | |
| Vegetable oil | 75˜55% |
| Solid additive | 15% |

Through special granulation process ( TW Patent No. 90133170), the ingredients are made into granules 1.0 mm˜3.0 mm in diameter with water content below 10%.

(3) Preparation of Oral Prophylactic Agent of the Present Invention

Mix the water-soluble formula ( in powder) and enteric-coated formula ( in granule) obtained in step (1) and (2) above in the ratio, preferably from 1:1˜1:10 by weight and most preferably 2:9.8 by weight, to obtain the oral prophylactic agent according to the present invention. This oral prophylactic agent may be further added into all kinds of food products and drinks.

### EXAMPLE 2

### Addition of Oral Prophylactic Agent of the Present Invention into Drinks

(1) Adding into Dairy Products

Add 1 gram of the oral prophylactic agent of the present invention into 1 liter of yogurt. The resulting mixture is readily drinkable.

(2) Adding in Water

Add 0.2 gram of prophylactic agent of the present invention into 200 ml of cold water, and add in proper amount of fructose or honey. The resulting mixture is readily drinkable.

What is claimed is:

1. An oral prophylactic agent against viral infection comprising water-soluble formula and/or enteric-coated formula, wherein at least one ingredient in said water-soluble formula or enteric-coated formula is selected from a group consisting of C-phycocyanin, allophycocyanin spirulina growth factor, and combinations thereof.

2. The oral prophylactic agent according to claim 1, wherein said water-soluble formula or enteric-coated formula is present separately or in combination.

3. An oral prophylactic agent against viral infection comprising the combination of water-soluble formula and enteric-coated formula, wherein the ratio of water-soluble formula to enteric-coated formula is 1:1˜1:10 by weight; said water-soluble formula or enteric-coated formula comprises at least one ingredient selected from a group consisting of C-phycocyanin, allophycocyanin, spirulina growth factor and combinations thereof.

4. The oral prophylactic agent according to claim 3, wherein the ratio of water-soluble formula to enteric-coated formula is 2:9.8 by weight.

5. An oral prophylactic agent against viral infection comprising the combination of water-soluble formula and enteric-coated formula, wherein both of which consist of the combination of C-phycocyanin, allophycocyanin and spirulina growth factor.

6. The oral prophylactic agent according to claim 5, wherein the ratio of water-soluble formula to enteric-coated formula is 1:1˜1:10 by weight.

7. The oral prophylactic agent according to claim 6, wherein the ratio of water-soluble formula to enteric-coated formula is 2:9.8.

8. The oral prophylactic agent according to claim 5, wherein the ingredients of said water-soluble formula are: 3˜45% C-phycocyanin, 1˜15% allophycocyanin, and 96˜40% spirulina growth factor.

9. The oral prophylactic agent according to claim 5, wherein said enteric-coated formula comprises said water-soluble formula, solid additive and vegetable oil in the percentage of 10˜30% water-soluble formula, 15% solid additive and 75˜55% vegetable oil.

10. The oral prophylactic agent according to claim 9, wherein the ingredients of water-soluble formula contained in said enteric-coated formula are 3˜45% C-phycocyanin, 1˜15% allophycocyanin, and 96˜40% spirulina growth factor.

11. The oral prophylactic agent according to claim 5, wherein the total concentration of C-phycocyanin and allophycocyanin ranges from 25 μg/mL to 2000 μg/mL.

12. The oral prophylactic agent according to claim 5, wherein the concentration of spirulina growth factor ranges from 1 μg/mL to 5000 μg/mL.

13. The oral prophylactic agent according to claim 5, wherein the ratio of phycocyanin to spirulina growth factor contained in said water-soluble formula is 2:7.

14. An oral prophylactic agent against viral infection, comprising either water-soluble formula or enteric-coated formula, either of which consists of C-phycocyanin, allophycocyanin and spirulina growth factor in the percentage of 3˜45% C-phycocyanin, 1˜15% allophycocyanin, and 96˜40% spirulina growth factor.

15. The oral prophylactic agent according to claim 14, wherein the ratio of phycocyanin to spirulina growth factor is 2:7.

16. The oral prophylactic agent according to claim 1, wherein said water-soluble formula is water-soluble powder, granule or any other water-soluble form.

17. The oral prophylactic agent according to claim 1, wherein said enteric-coated formula can be enteric-coated granule, tablet, capsule or any other enteric-coated form.

18. The oral prophylactic agent according to claim 13 or 15, wherein said phycocyanin refers to C-phycocyanin and allophycocyanin.

19. An oral composition for prevention of viral infection, comprising the combination of oral prophylactic agent according to claim 1 and food or drink.
